(19) United States
(12) Patent Application Publication
(10) Pub. No.: US 2026/0301968 A1
(43) Pub. Date: Oct. 1, 2026

(54) METHOD FOR PREDICTING AND TREATING THE LIKELIHOOD OF RELAPSE IN A PATIENT WITH DEPRESSION WHO HAS RECEIVED ANTIDEPRESSANT THERAPY, AND A DIAGNOSTIC AND THERAPEUTIC KIT FOR USE IN THE METHOD

(71) Applicant: CHONNAM NATIONAL UNIVERSITY HOSPITAL, Gwangju (KR)

(72) Inventors: JAE MIN KIM, Gwangju (KR); HEE JU KANG, Gwangju (KR)

(21) Appl. No.: 19/634,342

(22) Filed: Mar. 31, 2026

(30) Foreign Application Priority Data
Mar. 31, 2025 (KR) 10-2025-0040886

(51) Int. Cl. G16H 50/30; G16H 20/10; G16H 20/30; G16H 20/70

(52) U.S. Cl. CPC G16H 50/30; G16H 20/10; G16H 20/30; G16H 20/70

(57) ABSTRACT

A method for predicting and treating relapse in a patient with depression who has received antidepressant treatment includes analyzing a biomarker contained in a biological sample obtained from the patient at a baseline time point, thereby predicting whether the patient—who exhibited an acute-phase treatment response at 12 weeks after antidepressant therapy—has a likelihood of relapse over a subsequent period of 3 months to 2 years, and, upon determining that the patient is at risk of relapse, performing a relapse-prevention treatment.

### CROSS-REFERENCE TO RELATED APPLICATION AND CLAIM OF PRIORITY

This application claims the benefit under 35 U.S.C. § 119 of Korean Patent Application No. 10-2025-0040886 filed on Mar. 31, 2025, in the Korean Intellectual Property Office, the entire disclosure of which is incorporated herein by reference for all purposes.

### BACKGROUND

### 1. Technical Field

The present invention relates to a technology for predicting and treating the likelihood of relapse in patients with depression following antidepressant therapy. More specifically, the invention provides a method for predicting and treating relapse in a patient with depression who has received antidepressant treatment, wherein the method includes analyzing a biomarker contained in a biological sample obtained from the patient at a baseline time point, thereby predicting whether the patient—who exhibited an acute-phase treatment response at 12 weeks after antidepressant therapy—has a likelihood of relapse over a subsequent period of 3 months to 2 years, and, upon determining that the patient is at risk of relapse, performing a relapse-prevention treatment. The invention further relates to a diagnostic and therapeutic kit for use in the method.

### 2. Background Art

Depression significantly contributes to global disability, with a substantial portion of its burden arising from the high rate of relapse. It is reported that over half of individuals who experience a first depressive episode will endure a subsequent one, with the likelihood of additional episodes increasing thereafter (American Psychiatric Association, 2000). As such, the prevention of relapse is crucial in the comprehensive management of depression.

Identifying biological markers that can predict relapse is essential for tailoring personalized treatment approaches, enabling targeted interventions for high-risk individuals. Accessible and cost-effective, peripheral blood biomarkers are particularly advantageous for routine clinical monitoring. Among these, markers related to the hypothalamic-pituitaryadrenal (HPA) axis, such as cortisol, have been extensively studied due to their critical role in stress response systems. Although elevated cortisol levels have been generally predictive of relapse (Appelhof et al. 2006; Pintor et al. 2009), exceptions do exist (Bockting et al. 2012), necessitating further exploration. Additionally, immune-related markers like high-sensitivity C-reactive protein (hsCRP) and pro-inflammatory cytokines tumor necrosis factor-alpha (TNFα), interleukin-1 beta (IL-1β), IL-6 have been widely researched, while the findings have been controversial (Copeland et al. 2012; Glaus et al. 2018).

Other biomarkers, while not directly linked to relapse, have been implicated in the onset and persistence of depression, including metabolic markers like leptin (Milaneschi et al. 2015) and ghrelin (Ricken et al. 2017); neurotransmitters such as serotonin (Dvojkovic et al. 2021; Holck et al., 2024); neurogenic markers like brain-derived neurotrophic factor (BDNF) (Kuhlmann et al. 2017; Zou et al., 2024); and nutritional indicators including folate and homocysteine (Kim et al. 2008).

Despite substantial research efforts, the predictive utility of individual biomarkers in clinical settings remains limited, often due to their insufficient predictive values.

Therefore, there is a need to develop novel biomarkers that are sufficiently effective as predictors of the likelihood of relapse following an acute-phase treatment response in patients with depression who have received antidepressant therapy.

### SUMMARY

The present inventors, based on extensive research, have found that the likelihood of relapse in patients with depression who exhibit a treatment response to acute-phase treatment, i.e., 12 weeks of antidepressant therapy, can be predicted and treated based on the concentration of specific biomarkers present in a biological sample obtained from the patient, thereby completing embodiments of the present invention.

Therefore, an aspect of the present invention is to provide a method for predicting and treating the likelihood of relapse in a patient with depression who has received antidepressant therapy, wherein, when a specific biomarker is present in a biological sample obtained from the patient at baseline at a concentration exceeding or below a predetermined cut off level, it has been experimentally confirmed that the likelihood of relapse increases in patients who exhibit an acute-phase treatment response following administration of an antidepressant, and wherein one or more biomarkers and corresponding cut off levels for predicting the likelihood of relapse after the acute-phase treatment response are identified. Accordingly, because the likelihood of relapse can be predicted at baseline, i.e., prior to initiation of treatment in patients who subsequently exhibit an acute-phase treatment response after antidepressant administration, patients at high risk of relapse can be more accurately identified, thereby enabling clinicians to adopt more personalized and effective approaches in selecting therapeutic agents and/or treatment modalities.

Another aspect of the present invention is to provide a diagnostic and therapeutic kit for predicting and treating the likelihood of relapse in a patient with depression who has received antidepressant therapy, having excellent clinical utility, wherein the likelihood of relapse can be predicted in patients who exhibit an acute-phase treatment response following antidepressant administration by measuring, at baseline, the concentrations of one or more biomarkers for predicting relapse selected from the group consisting of cortisol, high-sensitivity C-reactive protein (hsCRP), tumor necrosis factor-α (TNF-α), and brain-derived neurotrophic factor (BDNF) in a biological sample obtained from the patient, and wherein, based on the prediction results, a personalized treatment strategy is provided for each patient.

Aspects of the present invention are not limited to the aspects mentioned hereinabove, and other aspects not mentioned will be clearly understood by those skilled in the art from the following description.

To achieve the aforementioned aspects of the present invention, an embodiment of the present invention first provides a method for predicting and treating a likelihood of relapse in a patient with depression who has received antidepressant therapy, the method including: measuring, in a biological sample obtained from the patient at baseline, a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF); determining the likelihood of relapse over a period of 3 months to 2 years following an acute-phase treatment response by comparing the measured concentration of the biomarker with a predetermined cut off level; and performing a relapse-prevention treatment when the patient is determined to have a likelihood of relapse based on the determination.

In a preferred embodiment, the predetermined cut off level for each biomarker is 10.62 μmol/L for cortisol, 0.56 mg/dL for high-sensitivity C-reactive protein (hsCRP), 0.58 μg/mL for tumor necrosis factor-α (TNF-α), and 23.30 ng/ml for brain-derived neurotrophic factor (BDNF), when the biomarker is cortisol, hsCRP, or TNF-α, the likelihood of relapse is determined to be present when the measured concentration of the biomarker exceeds the corresponding cut off level, and when the biomarker is BDNF, the likelihood of relapse is determined to be present when the measured concentration of the biomarker is less than the corresponding cut off level.

In a preferred embodiment, when the number of the biomarkers is one, the likelihood of relapse increases by 1.71 times relative to a reference level, and when the number of the biomarkers is four, the likelihood of relapse increases by 8.28 times relative to the reference level.

In a preferred embodiment, performing the relapse-prevention treatment includes maintenance therapy for at least 24 months, wherein the maintenance therapy includes either a first-stage treatment or a second-stage treatment determined based on whether the patient maintains an acute-phase treatment response or remission, as assessed through symptom re-evaluation at intervals of 3 to 6 months.

In a preferred embodiment, the first-stage treatment includes maintaining an ongoing treatment while the patient maintains an acute-phase treatment response or remission, and the second-stage treatment includes, in a state in which residual symptoms are present in the patient, performing one or more treatments selected from the group consisting of intensifying the first-stage treatment conditions, biomarker-targeted therapy, electroconvulsive therapy (ECT), repetitive transcranial magnetic stimulation (rTMS), and esketamine nasal spray, based on a clinician's judgment.

In a preferred embodiment, in the first-stage treatment, the ongoing treatment includes, in addition to continued administration of an antidepressant, one or more of pharmacological augmentation therapy, evidence-based psychotherapy, and psychosocial supportive interventions.

An embodiment of the present invention includes a method for predicting and treating a likelihood of relapse in a patient with depression who has received antidepressant therapy, the method including: measuring, in a biological sample obtained from the patient at baseline, concentrations of four biomarkers for predicting relapse, consisting of cortisol, high-sensitivity C-reactive protein (hsCRP), tumor necrosis factor-α (TNF-α), and brain-derived neurotrophic factor (BDNF); assigning a reference score to each of the four biomarkers by comparing the measured concentrations with predetermined cut off levels, wherein the predetermined cut off levels are 10.62 μmol/L for cortisol, 0.56 mg/dL for hsCRP, 0.58 μg/mL for TNF-α, and 23.30 ng/ml for BDNF, wherein, for cortisol, hsCRP, and TNF-α, a score of 1 is assigned when the measured concentration exceeds the corresponding cut off level, and a score of 0 is assigned when the measured concentration is equal to or less than the corresponding cut off level, and wherein, for BDNF, a score of 1 is assigned when the measured concentration is less than the corresponding cut off level, and a score of 0 is assigned when the measured concentration is equal to or greater than the corresponding cut off level; calculating a continuous multi-biomarker score according to Equation 1: continuous multi-biomarker score=0.392×A+0.817×B+0.603×C+0.499×D, wherein A is a reference score for cortisol, B is a reference score for hsCRP, C is a reference score for TNF-α, and D is a reference score for BDNF; determining the likelihood of relapse by classifying the calculated score into one of quartiles 1 to 4; and performing a relapse-prevention treatment when the patient is determined to have a likelihood of relapse based on the determination.

In a preferred embodiment, the continuous multi-biomarker score is classified into quartiles such that a score of 0.000 to 0.576 is assigned to a first quartile, a score of 0.577 to 1.125 is assigned to a second quartile, a score of 1.126 to 1.701 is assigned to a third quartile, and a score of 1.702 to 2.311 is assigned to a fourth quartile.

In a preferred embodiment, the likelihood of relapse is 24.7% when classified in the first quartile, increases by 1.78 times relative to the first quartile when classified in the second quartile, increases by 2.43 times relative to the first quartile when classified in the third quartile, and increases by 5.31 times relative to the first quartile when classified in the fourth quartile.

In a preferred embodiment, performing the relapse-prevention treatment includes maintenance therapy for at least 24 months, wherein the maintenance therapy includes either a first-stage treatment or a second-stage treatment determined based on whether the patient maintains an acute-phase treatment response or remission, as assessed through symptom re-evaluation at intervals of 3 to 6 months.

In a preferred embodiment, the first-stage treatment includes maintaining an ongoing treatment while the patient maintains an acute-phase treatment response or remission, and the second-stage treatment includes, in a state in which residual symptoms are present in the patient, performing one or more treatments selected from the group consisting of intensifying the first-stage treatment conditions, biomarker-targeted therapy, electroconvulsive therapy (ECT), repetitive transcranial magnetic stimulation (rTMS), and esketamine nasal spray, based on a clinician's judgment.

In a preferred embodiment, in the first-stage treatment, the ongoing treatment includes, in addition to continued administration of an antidepressant, one or more of pharmacological augmentation therapy, evidence-based psychotherapy, and psychosocial supportive interventions.

Another embodiment of the present invention includes a diagnostic and therapeutic kit for predicting and treating a likelihood of relapse in a patient with depression who has received antidepressant therapy, the kit including: a biomarker measurement unit configured to measure a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF) in a biological sample obtained from the patient; a relapse likelihood determination guide configured to determine a likelihood of relapse over a period of 3 months to 2 years following an acute-phase treatment response based on the concentration of the biomarker measured by the biomarker measurement unit; and a treatment guide for a clinician configured to provide a treatment strategy for a patient determined to have a likelihood of relapse according to the relapse likelihood determination guide.

In a preferred embodiment, the biomarker measurement unit includes one or more selected from the group consisting of a high-sensitivity bead-based panel utilizing an antigen-antibody reaction, an enzyme-linked immunosorbent assay (ELISA), an electrochemiluminescence immunoassay (ECLIA), and an enzymatic method.

In a preferred embodiment, the relapse likelihood determination guide includes predetermined cut off levels for each biomarker, likelihood of relapse values according to a number of biomarkers, a calculation formula for a continuous biomarker score, quartile positions determined based on a calculated continuous multi-biomarker score, and likelihood of relapse values corresponding to the quartile positions.

In a preferred embodiment, the treatment guide for the clinician includes a relapse-prevention treatment, wherein the relapse-prevention treatment includes maintenance therapy for at least 24 months, and wherein the maintenance therapy includes either a first-stage treatment or a second-stage treatment determined based on whether the patient maintains an acute-phase treatment response or remission, as assessed through symptom re-evaluation at intervals of 3 to 6 months.

In a preferred embodiment, the first-stage treatment includes maintaining an ongoing treatment while the patient maintains an acute-phase treatment response or remission, and the second-stage treatment includes, in a state in which residual symptoms are present in the patient, performing one or more treatments selected from the group consisting of intensifying the first-stage treatment conditions, biomarker-targeted therapy, electroconvulsive therapy (ECT), repetitive transcranial magnetic stimulation (rTMS), and esketamine nasal spray, based on a clinician's judgment.

In a preferred embodiment, in the first-stage treatment, the ongoing treatment includes, in addition to continued administration of an antidepressant, one or more of pharmacological augmentation therapy, evidence-based psychotherapy, and psychosocial supportive interventions.

According to the above-described embodiments of the present invention, it has been confirmed that one or more biomarkers selected from the group consisting of cortisol, hsCRP, and TNF-α, when present at concentrations exceeding predetermined levels, or BDNF when present at a concentration below a predetermined level in a biological sample obtained from a patient with depression at baseline, can serve as biomarkers for predicting the likelihood of relapse in patients who exhibit an acute-phase treatment response following antidepressant administration. Accordingly, an embodiment of the present invention provides biomarkers capable of relatively accurately predicting the likelihood of relapse over a period of 3 months to 2 years following the acute-phase treatment response, even at baseline before initiation of treatment for depression.

In addition, according to the method for predicting and treating the likelihood of relapse in a patient with depression who has received antidepressant therapy of an embodiment of the present invention, by identifying one or more biomarkers and corresponding cut off levels for predicting relapse, it is possible to predict, at baseline prior to initiation of treatment, the likelihood of relapse in patients who will exhibit an acute-phase treatment response following antidepressant administration, thereby contributing to a clinician's decision-making process regarding therapeutic agents and/or treatment strategies.

Furthermore, the diagnostic and therapeutic kit for predicting and treating the likelihood of relapse in a patient with depression who has received antidepressant therapy according to an embodiment of the present invention enables prediction of relapse in patients who exhibit an acute-phase treatment response following antidepressant administration by measuring the concentrations of one or more of the aforementioned biomarkers for predicting relapse in a biological sample obtained from the patient at baseline, and, based on the prediction results, provides a personalized treatment strategy for each patient, thereby demonstrating clinical utility.

Effects of the present invention are not limited to the effects mentioned hereinabove, and other effects not mentioned will be clearly understood by those skilled in the art from the following description.

### BRIEF DESCRIPTION OF THE DRAWINGS

FIG. 1 is a flowchart illustrating one embodiment of a method for predicting and treating a likelihood of relapse in a patient with depression who has received antidepressant therapy according to an embodiment of the present invention.

FIG. 2 is a flowchart illustrating one embodiment of performing a relapse-prevention treatment, which is a final step shown in FIG. 1.

### DETAILED DESCRIPTION

The terminology used herein is for the purpose of describing particular embodiments only and is not intended to limit the present invention. As used herein, singular forms include plural forms as well, unless the context clearly indicates otherwise. In the present application, the terms “comprise,” “include,” or “have” are intended to specify the presence of stated features, numbers, steps, operations, elements, components, parts, or combinations thereof, but do not preclude the presence or addition of one or more other features, numbers, steps, operations, elements, components, parts, or combinations thereof.

The terms “first,” “second,” and the like may be used to describe various elements, but such elements should not be limited by these terms. These terms are only used to distinguish one element from another. For example, a first element may be termed a second element without departing from the scope of the present invention, and similarly, a second element may be termed a first element.

Unless otherwise defined, all terms used herein, including technical and scientific terms, have the same meaning as commonly understood by a person having ordinary skill in the art to which the present invention pertains. Terms such as those defined in commonly used dictionaries should be interpreted as having meanings consistent with their meanings in the context of the relevant art and should not be interpreted in an idealized or overly formal sense unless expressly so defined herein.

In interpreting elements, it is understood that a range of error is included even in the absence of an explicit description thereof. In particular, when terms of degree such as “about” and “substantially” are used, they are to be interpreted as encompassing inherent manufacturing and material tolerances, and thus may be construed as having meanings at or near the stated values.

With respect to temporal relationships, when terms such as “after,” “subsequent to,” “following,” and “before” are used to describe a temporal sequence, such terms are intended to include non-consecutive relationships unless the terms “immediately” or “directly” are expressly used.

As used herein, the term “acute-phase treatment response” refers to a case in which a Hamilton Depression Rating Scale (HAMD) score measured after 12 weeks of antidepressant treatment in a patient with depression is less than 14.

As used herein, the term “remission” refers to a case in which a Hamilton Depression Rating Scale (HAMD) score measured in a patient with depression is 7 or less.

Accordingly, as used herein, the “acute-phase treatment response” does not necessarily indicate that the patient with depression has reached medically defined “remission” through antidepressant treatment, but may refer to a state in which the antidepressant treatment has produced a meaningful therapeutic effect such that the symptoms are alleviated to a level at which the patient perceives little to no symptoms.

As used herein, the term “baseline” refers to a time point at which an initial clinical visit for administration of an antidepressant to a patient with depression is performed.

As used herein, the term “diagnosis (or testing)” refers to identifying the presence or characteristics of a pathological condition. In the context of the present invention, “diagnosis” refers to determining or predicting, based on an in vitro analysis of a biological fluid obtained from a patient with depression at baseline, whether the patient—who exhibits an acute-phase treatment response at 12 weeks after initiation of antidepressant therapy—has a likelihood of relapse over a period of 3 months to 2 years thereafter.

As used herein, the term “biomarker” refers to a substance that can indicate a disease state. In the context of the present invention relating to diagnosing a likelihood of relapse over a period of 3 months to 2 years following an acute-phase treatment response, the “biomarker” refers to one or more selected from the group consisting of cortisol, high-sensitivity C-reactive protein (hsCRP), and tumor necrosis factor-α (TNF-α) having concentrations exceeding corresponding cut off levels, or brain-derived neurotrophic factor (BDNF) having a concentration less than a corresponding cut off level. The greater the number of such biomarkers present in a patient with depression at baseline, the higher the likelihood of relapse over a period of 3 months to 2 years following an acute-phase treatment response to antidepressant administration, as compared to a reference level in which no biomarker is present.

As used herein, the term “cut off level” refers to a relative level or an absolute level of a biomarker for predicting relapse in depression, measured from a patient with depression at baseline, which is determined so as to distinguish subjects having a likelihood of relapse over a period of 3 months to 2 years following an acute-phase treatment response. The term “cut off level” and “cut-off value” may be used interchangeably herein. The cut off level may be expressed, in the case of a relative level, as a fold difference, or, in the case of an absolute level, for example, as a concentration value. As described herein, depending on the type of biomarker, a value less than or greater than the cut off level may be regarded as determining the likelihood of relapse over a period of 3 months to 2 years following an acute-phase treatment response at baseline.

As used herein, the term “reference level” refers to a state in which no biomarker is present in a biological sample obtained from a patient with depression at baseline, that is, a state in which the concentrations of cortisol, hsCRP, and TNF-α among the four aforementioned biomarkers are all equal to or less than their respective cut off levels, and the concentration of BDNF is equal to or greater than its corresponding cut off level.

As used herein, the term “prediction” refers to identifying whether a subject who has exhibited an acute-phase treatment response following antidepressant administration has a low or high likelihood of relapse over a period of 3 months to 2 years.

As used herein, the term “biological sample” includes various types of samples obtained from a subject and may be used in diagnostic or monitoring analyses. Biological fluid samples include blood, cerebrospinal fluid (CSF), urine, and other biological fluid samples derived from the subject.

If necessary, the samples may be pretreated, for example, for concentration and separation.

As used herein, the term “blood” includes whole blood, serum, and plasma.

As used herein, the term “patient” refers to a mammal, preferably a human, and the terms “patient”, “subject” and “individual” may be used interchangeably herein.

Hereinafter, the technical configuration of the present invention will be described in detail with reference to the accompanying drawings and preferred embodiments.

However, the present invention is not limited to the embodiments described herein and may be embodied in various other forms. Throughout the specification, like reference numerals refer to like elements. In describing the present invention, detailed descriptions of related known configurations or functions may be omitted when they are deemed to unnecessarily obscure the subject matter of the present invention.

The technical feature of the present invention lies in confirming that, when one or more biomarkers selected from the group consisting of cortisol, high-sensitivity C-reactive protein (hsCRP), and tumor necrosis factor-α (TNF-α) are present at concentrations exceeding corresponding cut off levels, or when brain-derived neurotrophic factor (BDNF) is present at a concentration less than a corresponding cut off level in a biological sample obtained from a patient with depression at baseline, such biomarkers can be used to predict a likelihood of relapse over a period of 3 months to 2 years in patients who exhibit an acute-phase treatment response following antidepressant administration. Based on this finding, the present invention provides a method for predicting and/or diagnosing a likelihood of relapse over a period of 3 months to 2 years in patients who exhibit an acute-phase treatment response following antidepressant administration, by using concentrations of the aforementioned biomarkers measured at baseline prior to administration of the antidepressant, and for treating the predicted likelihood of relapse, as well as a diagnostic and therapeutic kit for predicting and treating the likelihood of relapse.

As described below, the present invention elucidates a correlation by relatively accurately predicting, at a baseline, a likelihood of relapse over a period of 3 months to 2 years in patients with depression who exhibit an acute-phase treatment response following antidepressant administration, based on whether one or more of cortisol, hsCRP, and TNF-α present in a biological sample obtained from the patient at baseline have concentrations exceeding corresponding cut off levels, or whether BDNF has a concentration less than a corresponding cut off level.

In addition, the present invention provides a technical significance in establishing a theoretical basis by which a clinician can persuade patients who are likely to relapse to continue antidepressant therapy without discontinuation, even when an acute-phase treatment response has been observed, based on the identified correlation. In other words, although most patients generally tend to discontinue antidepressant medication early once an acute-phase treatment response is observed, and although continuation of antidepressant therapy is determined at the clinician's discretion, in the absence of other events such as the occurrence of residual symptoms after the acute-phase treatment response, clinicians have conventionally lacked objective grounds to persuade patients to continue treatment and have had to rely on clinical experience. However, according to the present invention, for patients determined to have a likelihood of relapse after the acute-phase treatment response, clinicians can objectively demonstrate such likelihood of relapse, and, based on this, can implement a relapse-prevention treatment for at least 24 months, thereby preventing relapse or enabling rapid and effective intervention even if relapse occurs.

Accordingly, the method for predicting and treating a likelihood of relapse in a patient with depression who has received antidepressant therapy according to the present invention may be classified into a first method in which one or more biomarkers for predicting relapse are measured at baseline, and a second method in which all four biomarkers are measured.

The first method may include: measuring, in a biological sample obtained from the patient at baseline, a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF); determining a likelihood of relapse over a period of 3 months to 2 years following an acute-phase treatment response by comparing the measured concentration of the biomarker with a predetermined cut off level; and performing a relapse-prevention treatment when the patient is determined to have a likelihood of relapse based on the determination.

The second method may include: measuring, in a biological sample obtained from the patient at baseline, concentrations of four biomarkers for predicting relapse, consisting of cortisol, high-sensitivity C-reactive protein (hsCRP), tumor necrosis factor-α (TNF-α), and brain-derived neurotrophic factor (BDNF); determining a likelihood of relapse over a period of 3 months to 2 years following an acute-phase treatment response by comparing the measured concentrations of the four biomarkers with predetermined cut off levels; and performing a relapse-prevention treatment when the patient is determined to have a likelihood of relapse based on the determination.

More specifically, in the first method, it is sufficient that one or more biomarkers for predicting relapse, selected from the group consisting of cortisol, hsCRP, TNF-α, and BDNF, are analyzed in the measuring, whereas the second method differs only in that all four biomarkers are analyzed in the measuring. The method for measuring concentrations of the biomarkers in the measuring may be the same in both methods, and the measurement method used in the measuring may be any known method useful for measuring the level or amount of biomarkers for predicting relapse following an acute-phase treatment response and for determining or diagnosing based on the measured level or amount.

In the present invention, the measurement method may be performed both in vitro and/or in vivo; however, preferably, the measurement method is an in vitro method based on a sample obtained from a subject and provided for analysis.

Next, in both the first method and the second method, the determining step is performed by comparing the concentrations of the biomarkers for predicting relapse measured in the measuring step with predetermined cut off levels. However, the first method considers only one or more of the measured biomarkers, whereas the second method considers all four measured biomarkers, and thus the two methods differ in this respect. Accordingly, each method will be described in sequence.

In the first method, the determining step may include determining that there is a likelihood of relapse over a period of 3 months to 2 years following an acute-phase treatment response when one or more of the measured biomarkers have concentrations less than or greater than predetermined cut off levels for each biomarker. That is, for cortisol, hsCRP, and TNF-α, when each measured concentration exceeds the corresponding cut off level, it is determined that there is a likelihood of relapse over a period of 3 months to 2 years following the acute-phase treatment response, whereas for BDNF, when the measured concentration is less than the corresponding cut off level, it is determined that there is a likelihood of relapse over the same period.

In the determining step, as the number of biomarkers for predicting relapse in depression increases, the likelihood of relapse increases relative to a reference level. Experimentally, it has been confirmed that when one biomarker is present, the likelihood of relapse increases by 1.71 times relative to the reference level; when two biomarkers are present, it increases by 2.70 times; when three biomarkers are present, it increases by 4.86 times; and when four biomarkers are present, it increases by 8.28 times. Here, the cut off levels are determined experimentally as described below and have different values depending on the type of biomarker. Specifically, when the biomarker is cortisol, the predetermined cut off level is 10.62 μmol/L; when the biomarker is hsCRP, the predetermined cut off level is 0.56 mg/dL; when the biomarker is TNF-α, the predetermined cut off level is 0.58 μg/mL; and when the biomarker is BDNF, the predetermined cut off level is 23.30 ng/mL.

Thus, the results obtained from the determining step of the first method demonstrate that, even when only one biomarker for predicting relapse is considered, the method is sufficiently meaningful in that it enables prediction of the likelihood of relapse at a level of at least about 1.7 times higher than the reference level. However, when all four biomarkers are considered, although the relapse rate at the reference level is 20% when evaluated based on the number of individual biomarkers, and increases to 24.7% when all four biomarkers are considered (as shown in Table 6), this does not necessarily represent a proportional increase in the predicted relapse rate relative to the reference level, but rather indicates an improvement in reliability. Accordingly, when the second method is employed, it is possible to more accurately predict the likelihood of relapse in patients with depression who have received antidepressant therapy at baseline, thereby obtaining more meaningful results.

In the second method, since all four biomarkers are considered, the determining step may include: assigning a reference score to each biomarker by comparing the measured concentrations with predetermined cut off levels, wherein, for cortisol, hsCRP, and TNF-α, a score of 1 is assigned when the measured concentration exceeds the corresponding cut off level and a score of 0 is assigned when the measured concentration is equal to or less than the corresponding cut off level, and wherein, for BDNF, a score of 1 is assigned when the measured concentration is less than the corresponding cut off level and a score of 0 is assigned when the measured concentration is equal to or greater than the corresponding cut off level;
calculating a continuous multi-biomarker score according to Equation 1:

continuous multi-biomarker score=0.392×A+0.817×B+0.603×C+0.499×D,
wherein A is the reference score for cortisol, B is the reference score for hsCRP, C is the reference score for TNF-α, and D is the reference score for BDNF; and
determining the likelihood of relapse by classifying the calculated score into one of quartiles 1 to 4.

Here, the predetermined cut off levels for each of the biomarkers for predicting relapse in depression are the same as described above. Accordingly, after assigning reference scores to each of the four biomarkers by comparing the measured concentrations with the corresponding cut off levels, a continuous multi-biomarker score may be calculated according to Equation 1. The calculated continuous multi-biomarker score is then classified into quartiles such that a score of 0.000 to 0.576 is assigned to a first quartile, a score of 0.577 to 1.125 is assigned to a second quartile, a score of 1.126 to 1.701 is assigned to a third quartile, and a score of 1.702 to 2.311 is assigned to a fourth quartile. Based on the quartile in which the score is positioned, that is, based on the continuous multi-biomarker score, the likelihood of relapse in depression can be predicted more accurately.

Specifically, when classified in the first quartile, the likelihood of relapse over a period of 3 months to 2 years following an acute-phase treatment response is 24.7%. When classified in the second quartile, the likelihood of relapse increases by 1.78 times relative to the first quartile; when classified in the third quartile, it increases by 2.43 times; and when classified in the fourth quartile, it increases by 5.31 times relative to the first quartile.

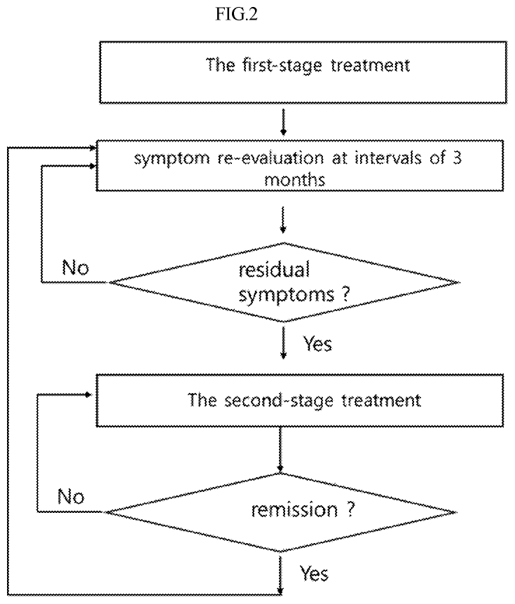

In both the first method and the second method, the step of performing a relapse-prevention treatment commonly includes maintenance therapy for at least 24 months. As illustrated in FIG. 2, the maintenance therapy may include a first-stage treatment or a second-stage treatment, determined based on whether the patient maintains remission, as assessed through symptom re-evaluation at intervals of 3 to 6 months.

The first-stage treatment includes maintaining an ongoing treatment in a state in which the patient maintains an acute-phase treatment response or remission, and the second-stage treatment may include, under a condition in which residual symptoms are present in the patient, an intensified treatment performed by reinforcing the conditions of the first-stage treatment, biomarker-targeted therapy, electroconvulsive therapy (ECT), repetitive transcranial magnetic stimulation (rTMS), and/or intranasal esketamine, as determined by the clinician.

In one embodiment, in the first-stage treatment, the ongoing treatment may include continued administration of an antidepressant, together with one or more selected from pharmacological augmentation therapy, evidence-based psychotherapy, and psychosocial support interventions. In this case, the continued administration of an antidepressant (i.e., basic treatment) refers to standard antidepressant therapy and may reduce the risk of relapse by approximately 50% compared to placebo (RR 0.52, 95% CI 0.46-0.59). Pharmacological augmentation therapy refers to a strategy of enhancing therapeutic efficacy by adding other drugs or agents to a standard antidepressant, that is, a first-line medication (e.g., selective serotonin reuptake inhibitors (SSRIs)), in patients who do not respond adequately to such first-line treatment, and may include, for example, lithium, thyroid hormones, atypical antipsychotics (e.g., olanzapine, aripiprazole, quetiapine), and certain anti-Parkinsonian agents. Evidence-based psychotherapy refers to a professional therapeutic approach in which treatments with demonstrated efficacy and safety through scientific research are applied in consideration of clinical expertise and patient characteristics, and may include cognitive behavioral therapy (CBT) and mindfulness-based cognitive therapy (MBCT). Psychosocial support interventions refer to approaches that combine professional treatment with the establishment of a supportive social environment to improve functional recovery and quality of life in patients with mental disorders, and may include psychoeducation, motivational interviewing (MI), and collaborative care models.

The second-stage treatment is performed when, upon symptom re-evaluation, the presence of residual symptoms or an insufficient treatment response is confirmed despite the first-stage treatment. If only one antidepressant has been used, an intensified treatment—by reinforcing the conditions of the first-stage treatment (e.g., combination of antidepressants, switching antidepressants, or switching/combining other augmentation agents)—may be considered first. If two or more antidepressants have already been used, electroconvulsive therapy (ECT), repetitive transcranial magnetic stimulation (rTMS), and intranasal esketamine may be considered.

When biomarker-targeted therapy is considered, it may be performed by regulating the concentrations of one or more of the biomarkers for predicting relapse after an acute-phase treatment response—namely cortisol, hsCRP, TNF-α, and BDNF—present in a patient with depression, such that cortisol≤10.62 μmol/L, hsCRP≤0.56 mg/dL, TNF-α≤0.58 μg/mL, and BDNF≥23.30 ng/ml are achieved.

In one embodiment, in order to regulate the cortisol level in the patient to cortisol≤10.62 μmol/L, hypothalamic-pituitary-adrenal (HPA) axis modulation therapy may be performed. Specifically, metyrapone may be administered to inhibit 11β-hydroxylase and thereby block cortisol synthesis (dosage: 500-1,000 mg/day), mifepristone may be administered as a glucocorticoid receptor antagonist (dosage: 300-1,300 mg/day), or ketoconazole may be administered to inhibit steroidogenic enzymes (dosage: 200-400 mg/day).

In another embodiment, anti-inflammatory therapy may be performed to regulate the hsCRP level to hsCRP≤0.56 mg/dL and/or the TNF-α level to TNF-α≤0.58 μg/mL. Specifically, celecoxib, a COX-2 inhibitor, may be administered (dosage: 200-400 mg/day); infliximab, a TNF-α monoclonal antibody, may be administered (dosage: 3-5 mg/kg); omega-3 eicosapentaenoic acid (EPA), which promotes anti-inflammatory eicosanoid synthesis, may be administered (dosage: 2≥g/day); or minocycline, which inhibits microglial activation, may be administered (dosage: 100-200 mg/day).

In another embodiment, neuroplasticity-enhancing adjunctive therapy may be performed to regulate the BDNF level to BDNF≥23.30 ng/ml. Specifically, aerobic exercise to increase BDNF gene expression may be employed (usage: ≤150 minutes/week, moderate intensity), or riluzole, which enhances AMPA receptor activity and upregulates BDNF, may be administered (dosage: 50-100 mg/day).

However, although biomarker-targeted therapy represents an important approach enabling personalized treatment, several considerations are required in its application. That is, since biomarker expression may change during disease progression or treatment, repeated monitoring is necessary, and the sensitivity and specificity of diagnostic methods, as well as standardization of preprocessing procedures, are also important. Furthermore, resistance may arise due to mutations in target molecules or activation of bypass signaling pathways, necessitating consideration of combination therapies or subsequent treatment strategies. In addition, potential unforeseen adverse effects due to expression in normal tissues cannot be excluded. Moreover, in order to ensure the efficacy and safety of biomarker-targeted therapy, comprehensive consideration should be given to clinical and regulatory aspects, including integration with companion diagnostics, compliance with regulatory requirements, and appropriate patient selection criteria.

As illustrated in FIG. 2, when a patient reaches remission through the second-stage treatment, the first-stage treatment may subsequently be performed. Conversely, if remission is not achieved, an intensified treatment may be carried out by switching to another treatment modality included in the second-stage treatment that has not yet been applied to the patient, or by combining two or more treatment modalities. In particular, as shown in FIG. 2, when the first-stage treatment is performed in a patient determined to have reached remission after the second-stage treatment, the ongoing treatment may include one or more of an augmentation therapy used in the second-stage treatment, electroconvulsive therapy (ECT), repetitive transcranial magnetic stimulation (rTMS), and intranasal esketamine maintenance therapy. Accordingly, in another embodiment of the first-stage treatment, the ongoing treatment may include the aforementioned ongoing treatment together with one or more selected from pharmacological augmentation therapy, evidence-based psychotherapy, and psychosocial support interventions.

Next, the diagnostic and therapeutic kit for predicting a likelihood of relapse in a patient with depression who is to receive antidepressant therapy according to the present invention is intended for use in determining a treatment strategy by identifying, at baseline prior to administration of an antidepressant, a likelihood of relapse over a period of 3 months to 2 years following an acute-phase treatment response. The kit includes: a biomarker measurement unit configured to measure a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF) in a biological sample obtained from the patient; a relapse likelihood determination guide configured to determine a likelihood of relapse over a period of 3 months to 2 years following an acute-phase treatment response based on the concentration of the biomarker measured by the biomarker measurement unit; and a treatment guide for a clinician configured to provide a treatment strategy for a patient determined to have a likelihood of relapse according to the relapse likelihood determination guide.

Here, the biomarker measurement unit may include one or more selected from the group consisting of a high-sensitivity bead-based panel utilizing an antigen-antibody reaction, an enzyme-linked immunosorbent assay (ELISA), an electrochemiluminescence immunoassay (ECLIA), and an enzymatic method. In one embodiment, cortisol may be measured using ECLIA, hsCRP may be measured using a monoclonal antibody specific to hsCRP, and TNF-α and BDNF may be measured using ELISA.

The relapse likelihood determination guide may include predetermined cut off levels for each biomarker, likelihood of relapse values according to a number of biomarkers, a calculation formula for a continuous biomarker score, quartile positions determined based on a calculated continuous multi-biomarker score, and likelihood of relapse values corresponding to the quartile positions.

The treatment guide for the clinician may include a relapse-prevention treatment, wherein the relapse-prevention treatment includes maintenance therapy for at least 24 months, and wherein the maintenance therapy includes a first-stage treatment or a second-stage treatment determined based on whether the patient maintains remission, as assessed through symptom re-evaluation at intervals of 3 to 6 months.

Here, a patient confirmed, by the measurement unit, to have one or more of cortisol, hsCRP, and TNF-α present at concentrations exceeding the corresponding cut off levels, or to have BDNF present at a concentration below the corresponding cut off level, may be determined to have a likelihood of relapse that is at least 1.7 times higher over a period of 3 months to 2 years following an acute-phase treatment response, as compared to a patient corresponding to a reference level.

In addition, the biological sample obtained from the patient with depression may be serum, and the biomarker measurement unit may be implemented as a microarray.

Meanwhile, the antidepressants used for evaluating the 12-week treatment response may include most known pharmaceutical agents, and, as described below, may specifically include bupropion, desvenlafaxine, duloxetine, escitalopram, fluoxetine, mirtazapine, paroxetine, sertraline, venlafaxine, and vortioxetine.

### Example

### 1. Study Outline and Design

The present invention is a key part of the MAKE BETTER (Biomarker discovery for Enhancing Antidepressant Treatment Effect and Response) initiative, aiming to formulate a predictive index for treatment outcomes using biomarkers in depressive disorders. The study framework has been detailed in a previously published protocol (Kang et al., 2018) and is registered under the identifier KCT0001332 at cris.nih.go.kr. Reflecting true clinical situations, recruitment was inclusive of various depression subtypes and comorbid physical conditions. Treatment was administered in a naturalistic manner, allowing flexibility in medication type, dosage, and regimen based on patient and clinician preferences, structured around predefined intervals and measurement points. During the initial 3-week phase of antidepressant monotherapy, subsequent adjustments were permitted at 3-week intervals in the acute phase and every 3 months in the continuation and maintenance phases. Data collection on biomarkers and other variables was conducted via a structured clinical report form by coordinators unaware of the treatment details, following training by psychiatric researchers. Ethical approval was granted by the Chonnam National University Hospital (CNUH) Institutional Review Board.

### 2. Participants

Recruitment occurred from March 2012 to April 2017 at the outpatient psychiatric department of CNUH, targeting patients initiating new antidepressant treatments for either first-onset or recurrent depressive symptoms. Enrollment was based on the intent to identify biomarkers predictive of antidepressant outcomes, requiring participants' consent to receive only antidepressant-based therapies. Inclusion criteria included adults over 18 years diagnosed with major depressive disorder (MDD), dysthymic disorder, or depressive disorder not otherwise specified, per Diagnostic and Statistical Manual of Mental Disorders, Fourth Edition (DSMIV) criteria (APA, 1994), with a Hamilton Depression Rating Scale (HAMD) (Hamilton, 1960) score of 14 or higher. Exclusions encompassed severe concurrent medical or psychiatric conditions, history of organic psychosis, and pregnancy, among others. Written informed consent was obtained from all participants and from a guardian for minors.

### 3. Baseline Evaluations

### (1) Biomarker Assessment

Participants were instructed to fast overnight prior to morning blood sampling and to rest quietly in a seated position for 25 to 45 minutes before blood collection. Blood samples (10 mL) were collected into dry tubes and immediately stored in a refrigerator at 2-4° C. for 3 to 6 hours. On the same day as blood sampling, centrifugation was performed at 4° C. for 15 minutes at 3000×g, and the resulting serum samples were immediately frozen at −80° C. in the clinical laboratory of CNUH. All laboratory measurements were performed at a Global Clinical Central Laboratory (Yongin, Republic of Korea) blinded to the clinical status of the patients. Samples were thawed immediately prior to measurement, and analyses were conducted simultaneously to avoid multiple freeze-thaw cycles and batch effects.

Based on a literature review and meta-review (Kennis et al., 2020), fourteen blood biomarkers representing six functional systems were selected. The blood biomarkers were measured using the following methods:

### (i) HPA Axis

Cortisol: measured using the Cobas Cortisol II electrochemiluminescence immunoassay (Roche, Vilvoorde, Belgium).

### (ii) Immune System

hsCRP: measured using the Tina-quant C-reactive protein (latex) high-sensitivity assay (Roche, Vilvoorde, Belgium).

TNF-α: measured using the QUANTIKINER HS ELISA human TNF-α immunoassay (R&D Systems, Minneapolis, USA).

IL-1β, IL-6, IL-4, IL-10: measured using a human high-sensitivity T-cell magnetic bead panel (EMD Millipore, Billerica, USA).

(iii) Metabolic System

Leptin: measured using a human leptin ELISA (BioVendor Laboratory Medicine, Inc., Modrice, Czech Republic).

Total ghrelin: measured using a GHRELIN (Total) radioimmunoassay kit (EMD Millipore, Billerica, USA).

Total cholesterol: measured using an L-type CHO M cholesterol oxidase method kit (Wako Pure Chemical Industries, Osaka, Japan).

### (iv) Neurotransmitter System

Serotonin: measured using a ClinRep high-performance liquid chromatography kit (Recipe, Munich, Germany).

### (v) Neurogenesis or Neuroplasticity

BDNF: measured using the QUANTIKINER ELISA Human BDNF Immunoassay (R&D Systems Inc., Minneapolis, USA).

### (vi) Nutritional System

Folate: measured using the Cobas Elecsys Folate III electrochemiluminescence immunoassay (Roche, Vilvoorde, Belgium).

Homocysteine: measured using the ARCHITECT Homocysteine 1L71 kit (Abbott, Wiesbaden, Germany).

The intra-assay and inter-assay coefficients of variation (CV) for the biomarkers are presented in Table 1 below.

TABLE 1

| | | Intra-assay | | Inter-assay | |
|---|---|---|---|---|---|
| | | data | | data | |
| | Detec- | Test | Mean | Test | Mean |
| | tion | times | CV | times | CV |
| | Limit | (n) | (%) | (n) | (%) |
| High-sensitivity C-reactive | 0.03 | 21 | 0.62 | 21 | 3.59 |
| protein, mg/L | | | | | |
| Tumor necrosis | 0.011 | 20 | 2.03 | 20 | 6.53 |
| factor-α, pg/mL | | | | | |
| Interleukin-1β, pg/mL | 0.14 | 16 | <5.00 | 12 | <15.00 |
| Interleukin-6, pg/mL | 0.11 | 16 | <5.00 | 12 | <20.00 |
| Interleukin-4, pg/mL | 1.12 | 16 | <5.00 | 12 | <15.00 |
| Interleukin-10, pg/mL | 0.56 | 16 | <5.00 | 12 | <20.00 |
| Leptin, ng/mL | 0.2 | 8 | 5.90 | 6 | 5.55 |
| Ghrelin, pg/mL | 93 | 30 | 6.4 | 30 | 16.3 |
| Total cholesterol, mg/dL | 3.86 | 21 | 0.67 | 21 | 1.5 |
| Brain derived neurotrophic | 0.02 | 20 | 5.00 | 40 | 9.00 |
| factor, ng/mL | | | | | |
| Serotonin, ng/mL | 1.0 | 20 | 3.00 | 100 | 4.00 |
| Corti sol, μg/dL | 0.054 | 84 | 2.16 | 84 | 3.02 |
| Folate, ng/mL | 1.2 | 84 | 5.08 | 84 | 5.28 |
| Homocysteine, μmol/L | 1.0 | 80 | 2.46 | 80 | 4.32 |

### (2) Socio-Demographic and Clinical Characteristics

The socio-demographic data collected included variables such as age, sex, educational years, marital status (currently married or not), living situation (alone or with others), religious observance (practicing or not), employment status (employed or unemployed), and monthly earnings (either above or below 2,000 USD). The clinical data encompassed a range of diagnostic categories for depressive disorders with specific qualifiers, including the onset age and illness duration, the number of depressive episodes experienced previously, the length of the current episode, family depression history, the presence of simultaneous physical conditions assessed through a questionnaire covering 15 different systems, smoking status, and body mass index (BMI). Various scales were applied to measure symptoms and functional status: the Hospital Anxiety and Depression Scale (HADS) for depressive and anxiety symptoms, divided into the depression subscale (HADS-D) and the anxiety subscale (HADS-A) (Zigmond and Snaith, 1983); the Social and Occupational Functioning Assessment Scale (SOFAS) for evaluating functional ability (American Psychiatric Association, 1994); the Life Experiences Survey (LES) for assessing stressful life events (Sarason et al., 1978); and the Alcohol Use Disorders Identification Test (AUDIT) for issues related to alcohol use (Saunders et al., 1993). A higher score on the HADS-D, HADS-A, LES, and AUDIT signifies more severe symptoms, while a lower SOFAS score indicates reduced functional capacity.

### 4. Stepwise Psychopharmacotherapy

Details of the overall treatment stages and strategies used in this invention are as follows. Prior to initiation of treatment, a comprehensive evaluation was performed for each patient, including clinical symptoms (e.g., presence of psychotic or anxiety symptoms), severity of illness, physical comorbidities and medication profile, and history of previous treatments. The minimum and maximum dosages of medications were determined in accordance with established treatment guidelines (Anderson et al., 2008; Bauer et al., 2013).

In the first treatment stage (Stage 1), patients received antidepressant monotherapy for 3 weeks based on the above clinical data and treatment guidelines (Bauer et al., 2013; Malhi et al., 2015; Kennedy et al., 2016). The antidepressants used included bupropion, desvenlafaxine, duloxetine, escitalopram, fluoxetine, mirtazapine, paroxetine, sertraline, venlafaxine, and vortioxetine.

Following Stage 1 monotherapy, subsequent treatment steps could be administered as needed at 3-week intervals during the acute treatment phase and at 3-month intervals during the continuation and maintenance phases. At the end of each stage, overall efficacy and tolerability were reviewed prior to proceeding to the next step based on rating scale assessments.

If improvement was insufficient (defined as less than a 30% reduction in the Hamilton Depression Rating Scale (HAMD) score compared to the previous stage) or intolerable adverse effects were observed, patients were guided to either remain in the current stage or proceed to the next-stage strategy, including switching to another antidepressant(S), augmentation with a non-antidepressant agent (A), combination with another antidepressant (C), or a multi-strategy approach (S+A; S+C; A+C; S+A+C).

Patients could also proceed to the next stage if sufficient improvement (≥30% reduction in HAMD score) was observed and adverse effects were absent or tolerable. Patient preference was prioritized in determining the treatment strategy in order to maximize medication adherence and treatment outcomes.

Antidepressants used for switching or combination included bupropion, desvenlafaxine, duloxetine, escitalopram, fluoxetine, mirtazapine, paroxetine, sertraline, venlafaxine, and vortioxetine. Augmentation agents included buspirone, lithium, triiodothyronine, and atypical antipsychotics such as aripiprazole, risperidone, olanzapine, quetiapine, and ziprasidone.

Because the number of patients entering Stage 5 or higher was small, treatment stages were categorized into Stages 1, 2, 3, and 4 (including Stage 5 or higher) for analysis.

In summary, treatment was conducted according to a stepwise protocol beginning with initial antidepressant monotherapy, with potential adjustments every 3 weeks during the acute treatment phase (at 3, 6, 9, and 12 weeks), and every 3 months during the continuation phase (6, 9, and 12 months) and maintenance phase (15, 18, 21, and 24 months), based on treatment response and adverse effects. Treatment stages were classified into Stages 1 to 4 for analysis.

Medication adherence was estimated based on pill counts at each visit, and adherence was defined as poor when the prescribed dose taken was less than 50% (Haynes et al., 2002).

### 5. Follow-Up Evaluations of the Relapse

Patients who responded to the initial 12-week acute treatment phase with a HAMD score of less than 14 were selected for relapse analysis. Based on their HAMD scores at the 12-week point, these patients were categorized into two groups: those in remission (HAMD<8) and those showing partial response (HAMD scores between 8 and 13). Relapse assessments were conducted starting from the 12-week point and subsequently every three months up to 24 months, with a permissible seven-day window for reassessment. A relapse was defined as a HAMD score of 14 or greater at any point during the 24-month follow-up period, aligning with definitions used in prior studies (Stewart et al., 1998; Rush et al., 2006).

### 6. Statistical Analysis

Socio-demographic and clinical characteristics at baseline, as well as treatment related characteristics during the follow-up were compared by relapse status using t-tests or ×2 tests where applicable. Variables significantly linked to relapse (P<0.05) and those potentially influencing relapse (Berwian et al. 2016), including considerations for collinearity, were used as covariates in subsequent adjusted analyses. The association of baseline serum biomarker levels with relapse status was assessed using Mann-Whitney U tests. For biomarkers reaching statistical significance (P<0.05), we established optimal cut-off points with corresponding sensitivities and specificities through area under the receiver operating curve (AUROC) analysis. Odds ratios and 95% confidence intervals for relapse status were determined using these cut-offs through pairwise logistic regression, adjusted for selected covariates. The impact of multiple biomarkers on relapse was analyzed in two ways. Initially, binary scores (0 for favorable, 1 for unfavorable) were assigned based on the optimal cut-offs for each significant biomarker. These scores were then aggregated, with total scores ranging from 0 to 4, where higher scores indicated more unfavorable conditions. The relationship between an increasing number of unfavorable biomarkers and relapse was examined through logistic regression, adjusting for covariates, with the group scoring 0 serving as the reference. Additionally, a continuous multi-biomarker score was calculated using a formula (detailed in the footnotes of Table 4) that incorporated the significant biomarkers and their respective beta coefficients derived from the logistic models. This score was then categorized into quartiles, and odds ratios for each quartile were calculated with the lowest quartile as the reference. Tests for linear trends were carried out across both the cumulative biomarker scores and the quartiles. All statistical procedures were performed using SPSS version 27.0.

### 7. Results

### (1) Recruitment Overview

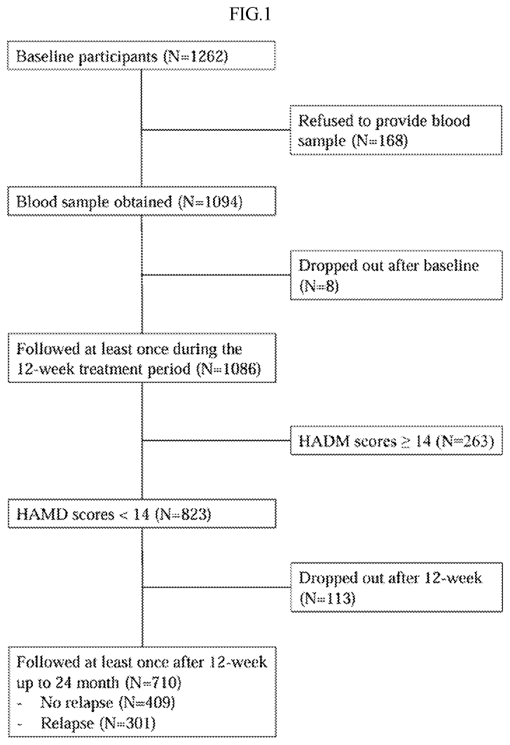

The patient flow according to treatment stages and strategies over the 24-month period is illustrated in FIG. 1. From the initial 1262 individuals assessed, 1094 (86.7%) provided blood samples and 1086 (86.1%) participated in at least one follow-up during the initial 12-week treatment period. There were no significant differences in baseline characteristics between the 1086 included patients and the 176 who were not included (all P>0.1). At the end of 12 weeks, 263 patients (24.2%) had a HAMD score of 14 or higher, whereas 823 had scores under 14. Of these 823, 710 (86.3%) were tracked through the 24 month follow-up period, forming the cohort for subsequent analysis. Dropout at 24 months correlated significantly with being unemployed and having lower SOFAS scores at baseline.

### (2) Relapse Status

Over the 24 months, the relapse rate was 42.4% (301 of 710 patients). A comparison of baseline and treatment-related characteristics by relapse status is provided in Table 2 and Table 3.

TABLE 2

| | No relapse | Relapse | Statistical | P- |
|---|---|---|---|---|
| | (N = 409) | (N = 301) | coefficients | value |
| Socio-demographic | | | | |
| characteristics | | | | |
| Age, mean (SD) years | 58.7 (13.9) | 56.8 (14.6) | t = +1.777 | 0.076 |
| Gender, N (%) female | 281 (68.7) | 212 (70.4) | χ² = 0.244 | 0.621 |
| Education, mean | 8.8 (4.9) | 9.3 (4.7) | t = −1.281 | 0.201 |
| (SD) years | | | | |
| Marital status, | 110 (26.9) | 83 (27.6) | χ² = 0.040 | 0.841 |
| N (%) unmarried | | | | |
| Living alone, N (%) | 63 (15.4) | 42 (14.0) | χ² = 0.289 | 0.591 |
| Religious affiliation, | 238 (58.2) | 173 (57.5) | χ² = 0.036 | 0.849 |
| N (%) | | | | |
| Unemployed status, | 98 (24.0) | 87 (28.9) | χ² = 2.199 | 0.138 |
| N (%) | | | | |
| Monthly income, | 242 (59.2) | 172 (37.1) | χ² = 0.293 | 0.588 |
| N (%) <2,000 USD | | | | |
| Clinical characteristics | | | | |
| Major depressive | 347 (84.8) | 260 (86.4) | χ² = 0.331 | 0.565 |
| disorder, N (%) | | | | |
| Melancholic feature, | 65 (15.9) | 50 (16.6) | χ^{2 =} 0.06 | 0.797 |
| N (%) | | | | |
| Atypical feature, | 27 (6.6) | 20 (6.6) | χ² = 0.001 | 0.982 |
| N (%) | | | | |
| Age at onset, mean | 53.1 (16.2) | 50.5 (15.9) | t = +2.095 | 0.036 |
| (SD) years | | | | |
| Duration of illness, | 5.9 (8.9) | 6.6 (9.6) | t = −0.892 | 0.373 |
| mean (SD) years | | | | |
| Number of depressive | 1.7 (3.9) | 2.3 (4.7) | t = −1.893 | 0.059 |
| episodes, mean (SD) | | | | |
| Duration of present | 7.1 (9.6) | 7.7 (10.6) | t = −0.765 | 0.445 |
| episode, mean | | | | |
| (SD) months | | | | |
| Family history of | 57 (13.9) | 50 (16.6) | χ² = 0.969 | 0.325 |
| depression, N (%) | | | | |
| Number of physical | 1.6 (0.5) | 1.5 (0.5) | t = +1.762 | 0.078 |
| disorders, mean (SD) | | | | |
| Current smoking, | 38 (9.3) | 33 (11.0) | χ² = 0.589 | 0.463 |
| N (%) | | | | |
| Body mass index, | 23.3 (3.0) | 23.3 (3.1) | t = +0.187 | 0.852 |
| mean (SD) kg/m² | | | | |

^{a} t-tests or χ² tests, as appropriate.
Bold style indicates statistical significance (P-value < 0.05).

TABLE 3

| | No relapse | Relapse | Statistical | P- |
|---|---|---|---|---|
| | (N = 409) | (N = 301) | coefficients | value |
| Assessment scales, mean | | | | |
| (SD) scores at baseline | | | | |
| Hospital Anxiety | 13.0 (4.0) | 13.9 (3.9) | t = −3.080 | 0.002 |
| & Depression | | | | |
| Scale-depression | | | | |
| subscale | | | | |
| Hospital Anxiety | 10.8 (3.8) | 12.3 (3.9) | t = −4.936 | <0.001 |
| & Depression | | | | |
| Scale-anxiety | | | | |
| subscale | | | | |
| Social and | 57.1 (7.5) | 55.9 (7.1) | t = +2.090 | 0.037 |
| Occupational Functional | | | | |
| Assessment Scale | | | | |
| Life Experiences Survey | 1.9 (13) | 2.1 (1.6) | t = −1.947 | 0.052 |
| Alcohol Use Disorders | 4.7 (7.8) | 5.9 (9.7) | t = −1.855 | 0.064 |
| Identification Test | | | | |
| Assessment scales, | | | | |
| mean (SD) scores | | | | |
| at 12 week follow-up | | | | |
| Hospital Anxiety | 5.5 (4.2) | 5.9 (4.2) | t = −1.378 | 0.169 |
| Depression | | | | |
| Scale-depression | | | | |
| subscale | | | | |
| Hospital Anxiety | 3.3 (3.3) | 3.9 (3.5) | t = −2.556 | 0.011 |
| & Depression | | | | |
| Scale-anxiety subscale | | | | |
| Social and | 74.6 (10.7) | 73.8 (10.1) | t = +1.068 | 0.286 |
| Occupational Functional | | | | |
| Assessment Scale | | | | |
| Treatment related | | | | |
| characteristics, N (%) | | | | |
| 12 week treatment | | | | |
| outcomes | | | | |
| Remission | 260 (63.6) | 179 (59.5) | χ² = 1.236 | 0.266 |
| Response without | 149 (36.4) | 122 (40.5) | | |
| remission | | | | |
| Treatment step | | | | |
| after 12 week | | | | |
| Step 1 | 150 (36.7) | 101 (33.6) | χ² = 8.314 | 0.040 |
| Step 2 | 147 (35.9) | 96 (31.9) | | |
| Step 3 | 86 (21.0) | 67 (22.3) | | |
| Step 4 | 26 (6.4) | 37 (12.3) | | |
| Poor medication adherence | 141 (34.5) | 151 (50.2) | χ² = 17.633 | <0.001 |
| after 12 week | | | | |

^{a} t-tests or χ² tests, as appropriate.
Bold style indicates statistical significance (P-value < 0.05).

Significant predictors of relapse included younger age at onset, higher initial scores on HADS-D and HADS-A, lower SOFAS scores, a greater number of treatment steps, and poorer medication adherence. Ten variables were identified as significant covariates for adjusted analyses, including demographic factors, baseline clinical scores, and treatment variables, factoring in past findings (Berwian et al. 2016) and variable collinearity.

### (3) Individual Biomarkers and Relapse Status

The associations between baseline serum biomarker levels and relapse are detailed in Table 4 [Baseline median (interquartile range) levels of serum biomarkers by relapse after 12 week acute treatment].

TABLE 4

| | No relapse | Relapse | U- | P- | Adjusted |
|---|---|---|---|---|---|
| | (N = 409) | (N = 301) | value^{a} | value^{a} | p-value^{b} |
| Cortisol, μg/dL | 10.2 (5.2) | 11.1 (6.2) | 72,270.5 | 0.027 | 0.036 |
| High-sensitivity | 0.3 (1.0) | 0.6 (0.9) | 87,425.5 | <0.001 | <0.001 |
| C-reactive | | | | | |
| protein, mg/L | | | | | |
| Tumor necrosis | 0.6 (0.4) | 0.7 (0.3) | 76,684.5 | <0.001 | 0.001 |
| factor-α, pg/mL | | | | | |
| Interleukin-1β, | 1.1 (0.6) | 1.1 (0.7) | 70,326.0 | 0.099 | 0.102 |
| pg/mL | | | | | |
| Interleukin-6, | 1.6 (17) | 1.6 (1.5) | 60,266.5 | 0.633 | 0.675 |
| pg/mL | | | | | |
| Interleukin-4, | 36.1 (38.7) | 37.4 (37.7) | 63,577.0 | 0.454 | 0.504 |
| pg/mL | | | | | |
| Interleukin-10, | 11.0 (10.3) | 10.6 (9.6) | 61,625.0 | 0.979 | 0.963 |
| pg/mL | | | | | |
| Leptin, ng/mL | 5.4 (6.4) | 5.7 (5.9) | 63,605.5 | 0.448 | 0.501 |
| Ghrelin, pg/ mL | 382.0 (188.5) | 374.0 | 60,690.0 | 0.749 | 0.681 |
| | | (165.5) | | | |
| Total | 175.0 (50.0) | 176.0 | 60,940.5 | 0.820 | 0.903 |
| cholesterol, | | (53.0) | | | |
| mg/dL | | | | | |
| Serotonin, | 70.8 (66.0) | 73.3 (67.6) | 61,410.5 | 0.957 | 0.918 |
| ng/mL | | | | | |
| Brain derived | 23.6 (8.4) | 21.3 (9.0) | 52,693.0 | 0.001 | 0.003 |
| neurotrophic | | | | | |
| factor, ng/mL | | | | | |
| Folate, ng/mL | 7.5 (6.6) | 7.7 (6.0) | 62,487.5 | 0.730 | 0.687 |
| Homocysteine, | 11.2 (4.7) | 10 8 (4.6) | 57,815.5 | 0.166 | 0.115 |
| μmol/L | | | | | |

^{a}Mann-Whitney U tests. ^{b}Adjustment for age, sex, age at onset, current smoking, body mass index, scores on Hospital Anxiety & Depression Scale-anxiety subscale, Social and Occupational Functional Assessment Scale, and Alcohol Use Disorders Identification Test, treatment step after 12 week, and poor medication adherence after 12 week. Bold style denotes statistical significance (p-value <0.05).

Relapse correlated with higher levels of cortisol, hsCRP, and TNF-α, and lower levels of BDNF, both before and after adjustment. AUROC analysis provided the optimal cut-offs for these biomarkers, delineated in Table 5 (Serum biomarker cut off values and risk of relapse after 12 weeks of acute treatment).

TABLE 5

| | Optimal | | | |
|---|---|---|---|---|
| | cut-off | OR (95% CI) | Sensitivity | Specificity |
| Cortisol | >10.62 μmol/L | 1.59 (1.11-2.07) | 54.2% | 53.5% |
| High-sensitivity | >0.56 mg/dL | 2.61 (1.92-3.54) | 62.8% | 60.6% |
| C-reactive | | | | |
| protein | | | | |
| Tumor necrosis | >0.58 pg/mL | 2.20 (1.62-3.00) | 62.8% | 56.7% |
| factor-α | | | | |
| Brain derived | <23.30 ng/ml | 1.66 (1.23-2.26) | 59.8% | 52.8% |
| neurotrophic | | | | |
| factor | | | | |

Optimal cut-off values were obtained from the receiver operating characteristic curve. Odds ratios (95% confidence intervals) [OR (95% CI)] were estimated by using logistic regression analyses after adjustment for age, sex, age at onset, current smoking, body mass index, scores on Hospital Anxiety & Depression Scale-anxiety subscale, Social and Occupational Functional Assessment Scale, and Alcohol Use Disorders Identification Test, treatment step after 12 week, and poor medication adherence after 12 week.

Post-adjustment logistic regression showed that exceeding these cut-off levels for cortisol, hsCRP, and TNF-α, and falling below for BDNF were independently associated with relapse.

### 4. Multiple Biomarkers and Relapse Status

Relapse risk in relation to biomarker numbers is described in the upper section of Table 6 (Number of serum biomarkers, quartiles of multi-biomarker scores, and risk of relapse after 12 week acute treatment). A progressive increase in relapse risk with an increasing number of unfavorable biomarkers was found, with statistical significance (P-value for trend<0.001). Compared to patients who did not exhibit any unfavorable biomarkers, those with four unfavorable biomarkers presented with a significantly higher relapse risk, with adjusted OR (95% CI) of 8.28 (3.60-18.45). In the lower section of Table 6, the data demonstrate a consistent increase in the risk of relapse across higher quartiles of multi-biomarker scores, with a significant trend (P-value for trend<0.001). The OR (95% CI) for relapse risk for patients in the highest quartile compared to those in the lowest quartile of multi-biomarker scores were 5.31 (3.33-8.52) after adjusting for confounding variables.

TABLE 6

| | | Relapse, | | P-value |
|---|---|---|---|---|
| | N | N (%) | OR (95% CI) | for trend |
| Number of | | | | |
| serum biomarkers^{a} | | | | |
| 0 | 55 | 11 (20.0) | Reference | <0.001 |
| 1 | 169 | 51 (30.2) | 1.71 (0.81-3.60) | |
| 2 | 250 | 101 (40.4) | 2.70 (1.34-5.49) | |
| 3 | 171 | 94 (55.0) | 4.86 (2.34-10.08) | |
| 4 | 65 | 44 (67.7) | 8.28 (3.60-18.45) | |
| Quartiles of | | | | |
| multi-biomarker | | | | |
| scores^{b} | | | | |
| 1 (lowest) | 166 | 41 (24.7) | Reference | <0.001 |
| 2 | 184 | 68 (37.0) | 1.78 (1.13-2.83) | |
| 3 | 190 | 84 (44.2) | 2.43 (1.53-3.82) | |
| 1 | 170 | 108 (63.5) | 5.31 (3.33-8.52) | |

Odds ratios (95% confidence intervals) [OR (95% CI)] were estimated by using logistic regression analyses after adjustment for age, sex, age at onset, current smoking, body mass index, scores on Hospital Anxiety & Depression Scale-anxiety subscale, Social and Occupational Functional Assessment Scale, and Alcohol Use Disorders Identification Test, treatment step after 12 week, and poor medication adherence after 12 week.

^{a}For calculating the number of serum biomarkers, 0 (favorable) or 1 (unfavorable) score from the optimal cut-offs of each significant biomarker (1 was allocated in case of high sensitivity C-reactive protein >0.56 mg/dL, tumor necrosis factor-α 10.62 μmol/L) was generated, and then summed scores were estimated ranging from 0 to 4, with higher scores indicating more unfavorable condition.

^{b}For calculating the continuous multi-biomarker scores, the following equations were used: (0.817×high-sensitivity C-reactive protein)+(0.603×tumor necrosis factor-α)+(0.499×brain derived neurotrophic factor)+(0.392×cortisol). Then, quartiles of the multi-biomarker scores were generated ranging from 1 to 4, with higher scores indicating more unfavorable condition.

### 8. Discussion

The principal findings of the present invention involving outpatients with depressive disorders, who were managed under a naturalistic and flexible treatment protocol, demonstrate a significant association between a combination of four serum biomarkers (cortisol, hsCRP, TNF-α, and BDNF) and the risk of relapse over a 24-month period. This relationship was observed to be dose-dependent, indicating that higher levels of these biomarkers collectively enhance the predictability of depression relapse.

Elevated cortisol, a crucial stress hormone in the HPA axis, can cause neuronal damage in critical brain regions like the hippocampus and desensitize glucocorticoid receptors, impairing stress regulation (Cowen, 2010). These effects suggest that high cortisol levels may worsen or trigger depressive episodes, increasing relapse risk. Previous studies on the relationship between immune markers and depression relapse have been mixed (Copeland et al. 2012; Glaus et al. 2018). In the present invention, significant associations were found with hsCRP and TNF-α, supporting the immune hypothesis for depression relapse. The mechanisms by which immune and inflammatory markers influence depression relapse involve chronic inflammation, which can disrupt neurobiological pathways. Elevated levels of inflammatory markers may lead to neuroinflammation, altering neurotransmitter systems, reducing neuroplasticity, and impairing stress response systems, thereby potentially triggering or worsening depressive episodes. (Calagua-Bedoya et al. 2024).

Research on biomarkers other than cortisol and immune markers for predicting depression relapse has been relatively limited. Our study found that low baseline levels of BDNF were predictive of depression relapse. BDNF is essential for neuron survival, growth, and differentiation, contributing to neuroplasticity, which is vital for learning and adapting to stress. Reduced levels of BDNF may decrease neuroplasticity, impair the brain's ability to respond to stress, and disrupt neurotransmitter systems involved in mood regulation, such as serotonin and dopamine, thereby increasing the risk of recurrent depression (Lima Giacobbo et al. 2019). Other markers related to metabolism, neurotransmitters, and nutrition investigated in this study did not show significant associations with depression relapse. Although there has not been extensive direct research on the relationship between these markers, including BDNF, and depression relapse, existing reports link them to responses to antidepressant treatments and the incidence or persistence of depression (Milaneschi et al. 2015; Ricken et al. 2017; Dvojkovic et al. 2021; Kuhlmann et al. 2017; Kim et al. 2008). Given these connections, additional research is required to replicate our findings and further explore the roles of these markers in the dynamics of depression.

The odds ratios (ORs) for the four significant biomarkers in predicting depression relapse ranged from 1.58 to 2.61 (Table 5). This invention's most significant finding was the substantially stronger and incremental predictive power when these biomarkers were combined. Specifically, the OR for patients with four unfavorable biomarkers was 8.28 compared to those without any, and the OR for the highest versus the lowest quartile of multi-biomarker scores was 5.31 (Table 6), marking a significant improvement over individual markers. Several mechanisms might explain the enhanced predictive power of combined biomarkers.

Firstly, this can be attributed to the multidimensional nature of depression, which involves complex interactions across various physiological systems, with each biomarker reflecting different aspects of depression's pathophysiology (Strawbridge et al. 2017).

Secondly, combining biomarkers from diverse biological pathways (e.g., hormonal, immune, metabolic) provides a more comprehensive assessment of depression's multifactorial nature, capturing a broader spectrum of potential dysregulations that might lead to relapse.

Thirdly, the interactions between different physiological systems may amplify the overall risk profile. For example, elevated cortisol may reflect stress response dysregulation and could exacerbate immune dysfunction, as indicated by inflammatory markers like hsCRP and TNF-α (Knezevic et al. 2023). These interactions potentially create a feedback loop that predicts relapse more strongly than any single marker.

Such combined analytic methods have been frequently used in longitudinal disease outcome studies (Wang et al., 2006). However, to our knowledge, this invention is the first to investigate the multimodal effects of blood biomarkers covering various functional systems on depression relapse.

The strengths of this invention include a larger sample size and a longer follow-up duration compared to prior biomarker studies. All participants were assessed using a structured research protocol and established, standardized scales. To our knowledge, this is the first study to evaluate the predictive value of a combination of biomarkers across different functional systems in forecasting depression relapse.

Depression, due to its frequent relapses, significant impacts individuals and healthcare systems. This invention demonstrated that a panel combining four serum biomarkers (cortisol, hsCRP, TNFα, and BDNF) could significantly improve the predictability of depression relapse. These findings have several clinical implications. By using a multimodal biomarker panel, clinicians can more accurately identify patients at high risk of relapse, potentially leading to more personalized and effective intervention strategies. Early identification of high-risk individuals could allow for tailored therapeutic approaches that address specific biological dysfunctions, thereby potentially reducing the frequency and severity of relapse episodes.

This invention also has future research implications. Firstly, additional studies are needed to validate these biomarkers across diverse populations and settings to confirm their general applicability and reliability. Secondly, research focused on understanding the mechanisms linking these biomarkers to depression relapse is necessary, as it would clarify the biological pathways involved and their interaction with treatment modalities. Lastly, integrating these biomarkers with newly emerging ones could further enhance predictive accuracy and provide a more comprehensive understanding of the biological foundation of depression. Our findings could serve ground for these future explorations.

Consequently, the present invention enables clinicians to predict the likelihood of depressive relapse over a period of 3 months to 2 years following an acute-phase treatment response, even prior to initiating antidepressant administration in patients with depression.

Accordingly, the present invention not only contributes to the clinical decision-making process of physicians in determining therapeutic strategies with respect to pharmacological agents and/or treatment modalities, but also holds significant promise as a potential tool that can substantially contribute to the clinical management of patients with depression

Although the present invention has been illustrated and described with the preferred embodiments as discussed hereinabove, the present invention is not limited to the aforementioned embodiments, and various changes and modifications can be made by those skilled in the art to which the present invention pertains, without departing from the spirit of the present disclosure.

What is claimed is:

1. A method for predicting and treating a likelihood of relapse in a patient with depression who has received antidepressant therapy, the method comprising:
measuring, in a biological sample obtained from the patient at baseline, a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF);
determining the likelihood of relapse over a period of 3 months to 2 years following an acute-phase treatment response by comparing the measured concentration of the one or more biomarkers with a predetermined cut off level; and
performing a relapse-prevention treatment when the patient is determined to have a likelihood of relapse based on the comparison.

2. The method according to claim 1, wherein, in the determining the likelihood of relapse,
the predetermined cut off level for each biomarker is 10.62 μmol/L for cortisol, 0.56 mg/dL for high-sensitivity C-reactive protein (hsCRP), 0.58 μg/mL for tumor necrosis factor-α (TNF-α), and 23.30 ng/mL for brain-derived neurotrophic factor (BDNF);
when the biomarker is cortisol, hsCRP, or TNF-α, the likelihood of relapse is determined to be present when the measured concentration of the biomarker exceeds the corresponding cut off level; and
when the biomarker is BDNF, the likelihood of relapse is determined to be present when the measured concentration of the biomarker is less than the corresponding cut off level.

3. The method according to claim 2, wherein, when the number of the biomarkers is one, the likelihood of relapse increases by 1.71 times relative to a reference level, and when the number of the biomarkers is four, the likelihood of relapse increases by 8.28 times relative to the reference level.

4. The method according to claim 1, wherein performing the relapse-prevention treatment comprises maintenance therapy for at least 24 months,
wherein the maintenance therapy comprises a first-stage treatment or a second-stage treatment determined based on whether the patient maintains an acute-phase treatment response or remission, as assessed through symptom re-evaluation at intervals of 3 to 6 months.

5. The method according to claim 4, wherein,
the first-stage treatment comprises maintaining an ongoing treatment while the patient maintains an acute-phase treatment response or remission, and
the second-stage treatment comprises, in a state in which residual symptoms are present in the patient, performing a treatment selected from the group consisting of intensifying the first-stage treatment conditions, biomarker-targeted therapy, electroconvulsive therapy (ECT), repetitive transcranial magnetic stimulation (rTMS), and esketamine nasal spray, or a combination thereof, based on a clinician's judgment.

6. The method according to claim 5, wherein, in the first-stage treatment, the ongoing treatment comprises, in addition to continued administration of an antidepressant, one or more of pharmacological augmentation therapy, evidence-based psychotherapy, and psychosocial supportive interventions.

7. A method for predicting and treating a likelihood of relapse in a patient with depression who has received antidepressant therapy, the method comprising:
measuring, in a biological sample obtained from the patient at baseline, concentrations of four biomarkers for predicting relapse, consisting of cortisol, high-sensitivity C-reactive protein (hsCRP), tumor necrosis factor-α (TNF-α), and brain-derived neurotrophic factor (BDNF);
assigning a reference score to each of the four biomarkers by comparing the measured concentrations with predetermined cut off levels,
wherein, for cortisol, hsCRP, and TNF-α, a score of 1 is assigned when the measured concentration exceeds the corresponding cut off level, and a score of 0 is assigned when the measured concentration is equal to or less than the corresponding cut off level,
wherein, for BDNF, a score of 1 is assigned when the measured concentration is less than the corresponding cut off level, and a score of 0 is assigned when the measured concentration is equal to or greater than the corresponding cut off level, and
wherein the predetermined cut off levels are 10.62 μmol/L for cortisol, 0.56 mg/dL for hsCRP, 0.58 μg/mL for TNF-α, and 23.30 ng/mL for BDNF;
calculating a continuous multi-biomarker score according to Equation 1:
continuous multi-biomarker score=0.392×A+0.817×B+0.603×C+0.499×D,
wherein A is the reference score for cortisol, B is the reference score for hsCRP, C is the reference score for TNF-α, and D is the reference score for BDNF;
determining the likelihood of relapse over a period of 3 months to 2 years following an acute-phase treatment response by classifying the calculated score into one of quartiles 1 to 4; and
performing a relapse-prevention treatment when the patient is determined to have a likelihood of relapse based on the determination.

8. The method according to claim 7, wherein, the continuous multi-biomarker score is classified into quartiles such that a score of 0.000 to 0.576 is assigned to a first quartile, a score of 0.577 to 1.125 is assigned to a second quartile, a score of 1.126 to 1.701 is assigned to a third quartile, and a score of 1.702 to 2.311 is assigned to a fourth quartile.

9. The method according to claim 8, wherein, the likelihood of relapse is 24.7% when classified in the first quartile, increases by 1.78 times relative to the first quartile when classified in the second quartile, increases by 2.43 times relative to the first quartile when classified in the third quartile, and increases by 5.31 times relative to the first quartile when classified in the fourth quartile.

10. The method according to claim 7, wherein performing the relapse-prevention treatment comprises maintenance therapy for at least 24 months,
wherein the maintenance therapy comprises a first-stage treatment or a second-stage treatment determined based on whether the patient maintains an acute-phase treatment response or remission, as assessed through symptom re-evaluation at intervals of 3 to 6 months.

11. The method according to claim 10, wherein, the first-stage treatment comprises maintaining an ongoing treatment while the patient maintains an acute-phase treatment response or remission, and
the second-stage treatment comprises, in a state in which residual symptoms are present in the patient, performing a treatment selected from the group consisting of intensifying the first-stage treatment conditions, biomarker-targeted therapy, electroconvulsive therapy (ECT), repetitive transcranial magnetic stimulation (rTMS), and esketamine nasal spray, or a combination thereof, based on a clinician's judgment.

12. The method according to claim 11, wherein, in the first-stage treatment, the ongoing treatment comprises, in addition to continued administration of an antidepressant, one or more of pharmacological augmentation therapy, evidence-based psychotherapy, and psychosocial supportive interventions.

13. A diagnostic and therapeutic kit for predicting and treating a likelihood of relapse in a patient with depression who has received antidepressant therapy, the kit comprising:
a biomarker measurement unit configured to measure a concentration of one or more biomarkers for predicting relapse selected from the group consisting of cortisol, high-sensitivity C-reactive protein (hsCRP), tumor necrosis factor-α (TNF-α), and brain-derived neurotrophic factor (BDNF) in a biological sample obtained from the patient;
a relapse likelihood determination guide configured to determine a likelihood of relapse over a period of 3 months to 2 years following an acute-phase treatment response based on the concentration of the biomarker measured by the biomarker measurement unit; and
a treatment guide for a clinician configured to provide a treatment strategy for a patient determined to have a likelihood of relapse according to the relapse likelihood determination guide.

14. The kit according to claim 13, wherein,
the biomarker measurement unit comprises one or more selected from the group consisting of a high-sensitivity bead-based panel utilizing an antigen-antibody reaction, an enzyme-linked immunosorbent assay (ELISA), an electrochemiluminescence immunoassay (ECLIA), and an enzymatic method.

15. The kit according to claim 13, wherein, the relapse likelihood determination guide comprises predetermined cut off levels for each biomarker, likelihood of relapse values according to a number of biomarkers, a calculation formula for a continuous multi-biomarker score, quartile positions determined based on the calculated continuous multi-biomarker score, and likelihood of relapse values corresponding to the quartile positions.

16. The kit according to claim 13, wherein, the treatment guide for the clinician comprises a relapse-prevention treatment,
wherein the relapse-prevention treatment comprises maintenance therapy for at least 24 months, and
wherein the maintenance therapy comprises a first-stage treatment or a second-stage treatment determined based on whether the patient maintains remission, as assessed through symptom re-evaluation at intervals of 3 to 6 months.

17. The method according to claim 16, wherein,
the first-stage treatment comprises maintaining an ongoing treatment while the patient maintains an acute-phase treatment response or remission, and
the second-stage treatment comprises, in a state in which residual symptoms are present in the patient, performing a treatment selected from the group consisting of intensifying the first-stage treatment conditions, biomarker-targeted therapy, electroconvulsive therapy (ECT), repetitive transcranial magnetic stimulation (rTMS), and esketamine nasal spray, or a combination thereof, based on a clinician's judgment.

18. The method according to claim 17, wherein, in the first-stage treatment, the ongoing treatment comprises, in addition to continued administration of an antidepressant, one or more of pharmacological augmentation therapy, evidence-based psychotherapy, and psychosocial supportive interventions.
